(19) United States
(12) Patent Application Publication
(10) Pub. No.: US 2021/0187161 A1
(43) Pub. Date: Jun. 24, 2021

(54) MEDICAL DEVICE COMPRISING GRAPHENE COATING

(71) Applicants: MASSACHUSETTS EYE AND EAR INFIRMARY, Boston, MA (US); MASSACHUSETTS INSTITUTE OF TECHNOLOGY, Cambridge, MA (US)

(72) Inventors: Miguel Gonzalez ANDRADES, Boston, MA (US); Paraskevi Evi KOLOVOU, Boston, MA (US); Mahomed Mehdi GOULAMALY, Boston, MA (US); Jing KONG, Winchester, MA (US); Tian MING, Cambridge, MA (US)

(73) Assignees: Massachusetts Eye and Ear Infirmary, Boston, MA (US); Massachusetts Institute of Technology, Cambridge, MA (US)

(21) Appl. No.: 16/093,474

(22) PCT Filed: Apr. 13, 2017
(86) PCT No.: PCT/US2017/027466

§ 371 Date: Oct. 12, 2018

Related U.S. Application Data
(60) Provisional application No. 62/322,561, filed on Apr. 14, 2016.

(51) Int. Cl. A61L 27/30

(52) U.S. Cl. CPC A61L 27/303; A61L 2430/16; A61L 27/16; A61L 2400/12; A61L 2400/18; A61L 2420/08

(57) ABSTRACT

The present invention provides a device or medical device comprising a graphene coating. Particularly, the graphene coating features substantially high transmittance, biointegrity and biocompatibility.

### BACKGROUND OF THE INVENTION

Corneal diseases are one of the most important causes of blindness in the third world (Pascolini et al., 2010, Br J Ophthalmol, 96, 614-8). Over 90% of people with bilateral corneal blindness live in developing countries, however, they are experiencing shortness of corneal transplant readiness (Oliva et al., 2012, Indian J Ophthalmol, 60, 423-7). Furthermore, even the transplanted eyes are at high risk of failure with traditional donor penetrating keratoplasty due to deep vascularization and/or limbal stem cell deficiency. Therefore, an economically affordable keratoprosthesis remains as the most promising possible treatment for corneal blindness in the developing and non-developed countries.

In this regard, an artificial keratoprosthesis, for example, Boston Keratoprosthesis (B-KPro), has been successfully used to treat corneal blindness or eyes that are not suitable for standard corneal transplantation (Aquavella et al., 2005, American Journal of Ophthalmology, 140, 1032-1038). The remaining challenges associated to the B-KPro may be postoperative complications (e.g. retroprosthetic membrane formation, sterile corneal necrosis and infectious endophthalmitis) (Aldave et al., 2012, Ophthalmology, 119, 1530-8). For example, the lack of integration of the B-KPro to the surrounding corneal tissue and the promotion of an inflammatory environment are in part the etiology of these complications, where the main component of the B-Kpro (polymethyl methacrylate—PMMA-) may play an important causal role (Dudenhoefer et al., 2003, Cornea, 22, 424-8; Harissi-Dagher et al., 2007, Cornea, 26, 564-8).

Meanwhile, in the related arts, other research groups have demonstrated that polymeric material such as polymethyl methacrylate (PMMA) does not promote cell attachment, but it stimulates a foreign body reaction associated to a chronic inflammatory response (Moure et al., 2012, InternationalJjournal of Oral and Maxillofacial Surgery, 41, 1296-303; Medeiros et al., 2014, International Journal of Oral and Maxillofacial Surgery, 43, 62-7; Vargas et al., 2014, Gerodontology; Linnola et al., 2003, Journal of Cataract and Refractive Surgery, 29, 146-52; Linnola et al., 2000b, Journal of Cataract and Refractive Surgery, 26, 1807-18; Linnola et al., 2000a, Journal of Cataract and Refractive Surgery, 26, 1792-806; Ruckhofer et al., 2000, Ophthalmology, 107, 2144-51).

Therefore, there is an urgent need to provide new materials and new designs for artificial corneas, or alternatively, artificial ocular devices that can improve biocompatibility and biointegration into human host tissue.

### SUMMARY OF THE INVENTION

The invention features a medical device comprising a substrate, and a graphene coating, wherein the graphene coating is substantially transparent and wherein the graphene coating has a thickness less than about 20 μm. In preferred aspects, the present invention provides a graphene coating and a medical device comprising the graphene coating.

In one preferred aspect, the present invention provides a medical device that comprises a substrate and a graphene coating. The graphene coating is disposed on at least one surface of the substrate. In particular, the graphene coating is substantially transparent and has a thickness less than about 20 μm. Preferably, the graphene coating may have a thickness less than about 20 μm, less than about 10 μm, less than about 5 μm, less than about 1 μm, less than about 500 nm, less than about 250 nm, less than about 125 nm, less than about 100 nm, or less than about 10 nm.

In certain embodiments, the graphene coating may be formed in a film. In certain embodiments, the graphene coating may comprise a single layer or multiple layers of graphene.

In certain embodiments, the medical device may be an ocular device. The graphene coating of the ocular device may have a light transmittance greater than about 80%, greater than about 85%, or greater than about 90%.

In certain exemplary embodiments, the substrate may comprise ceramics, polymer, composite, and mixtures thereof. In certain exemplary embodiments, the substrate may be formed in a plane, a disc, a ring, a semi-ring, a cylinder, a sphere, a semi-sphere or any combinations thereof. Preferably, the substrate may have a transmittance greater than about 80%.

In certain exemplary embodiments, the medical device may further comprise at least one of an external device, a sensor, a circuit, and a central processing unit (CPU), screen-based device, antennae, near field communication circuit, and wireless power source. In certain exemplary embodiments, the graphene coating may comprise a sensor unit. In certain exemplary embodiments, the graphene coating may comprise an ion, a biomolecule, a synthetic compound or a biomarker. In certain exemplary embodiments, the graphene coating may comprise a microchip or a transmitter that is connected to an external device. Such devices are useful to detect ocular parameters for diagnostic purposes, e.g., for detection of eye pressure.

In one preferred aspect, the present invention provides a method of manufacturing a medical device as described herein. The method may comprise depositing a graphene coating on a substrate, and in particular, the coated graphene is substantially transparent and suitably has a thickness less than about 20 μm. For example, the coated graphene may have a thickness less than about 20 μm, less than about 10 μm, less than about 5 μm, less than about 1 μm, less than about 500 nm, less than about 250 nm, less than about 125 nm, less than about 100 nm, or less than about 10 nm, as described herein. Preferably, the graphene is deposited by chemical vapor deposition (CVD). Alternatively, the graphene may be deposited by spraying an ink composition comprising graphene.

Further provided is a method of manufacturing a prosthesis that may comprise coating a substrate of the prosthesis with graphene, and the graphene coating is substantially transparent and has a thickness less than about 20 μm as described herein.

Still further provided is a method of manufacturing an ocular device that may comprise coating a substrate of the ocular device with graphene coating, and the graphene coating is substantially transparent and has a thickness less than about 20 μm as described herein. In certain exemplary embodiments, the ocular device is a keratoprosthesis, intrastromal corneal ring segment, and corneal inlays, a glaucoma valve, iris prosthesis, intraocular lens, scleral substitute, or retinal implant, but the examples are not limited thereto.

In one preferred aspect, the present invention provides a method of promoting proliferation of a cell or adhesion of a cell before or after implanting a prosthesis or a device to a subject, which may comprise coating the prosthesis or device with graphene as described herein. In certain embodiments, the cell may be a host cell or host tissue, or an allogenic or xenogeneic cell. For example, the cell comprises a human corneal limbal cell. The method encompasses promoting proliferation or adhesion of a cell such as a human limbal epithelial stem cell, a human corneal epithelial cell, or a human retinal pigment epithelial cell. In some embodiments, the contacting step occurs prior to implanting the device into a subject. In alternative embodiments, the contacting step occurs prior to implanting the device into a subject, after implantation, or both before and after implantation into a subject. In certain embodiments, the prosthesis may be an ocular device which may include a keratoprosthesis, an intrastromal corneal ring segment, a corneal inlay, a glaucoma valve, iris prosthesis, intraocular lens, scleral substitute, or retinal implant, but the examples are not limited thereto.

Further provided is a method of promoting biointegration of a prosthesis or a device to a subject. Such as method of promoting biointegrating of a prosthesis or a device to a subject is carried out by administering to a bodily tissue the prosthesis or device, the prosthesis or device being coated with graphene, wherein the graphene coated on the prosthesis or device has a thickness less than about 20 μm.

Further provided is a method of drug delivery comprising providing a medical device, as described herein, which particularly comprises the graphene film as described herein.

Publications, U.S. patents and applications, and all other references cited herein, are herby incorporated by reference. Other aspects of the invention are disclosed infra.

### DEFINITIONS

The “medical device”, as used herein, refers to a device subject to be used in a subject or a human body, or as being connected thereto for therapeutic or treatment purpose. The medical device can be, replace or function a part or portion of human body, such as organs, limbs, brain, muscle, bones, eyes and the like.

The term “graphene”, as used herein, refers to a thin layer of pure carbon material formed in a two-dimensional lattice. Graphene can exist in a sheet-like single layer or can be stacked in multiple layers. Graphene of the present invention can be deposited as coating layer and may be doped or include impurities, such other atoms or molecules, between stacks thereof or on surfaces thereof inserted during deposition process.

The term “substrate”, as used herein, refers to a base material that forms a structure and a shape of the device. Materials for the substrate may not be particularly limited, and the exemplary material may include ceramic, composite, polymer, metal, and the like.

The term “transmittance”, as used herein, refers to a ratio of penetrating or transmitting radiant energy through an object to received radiant energy. The transmittance can be defined in various ranges of radiant energy or radiant wavelengths, such as infrared radiation, visible light, and ultraviolet radiation. For example, human eye can typically detect and respond to visible light of which the wavelengths range from about 390 to 700 nm. Accordingly, the transmittance or the light transmittance, as used herein, can be defined at visible light wavelengths, e.g. from about 390 to 700 nm.

The term “transparent” or “substantially transparent”, as used herein, means to be transmitting radiation waves, particularly visible light, without significant reflection or scattering, such that the transparent object or substantially transparent object can be visibly clear and can be seen through. For example, the substantially transparent can be interpreted as to have a transmittance at least greater than about 50%, at least greater than about 60%, at least greater than about 70%, at least greater than about 80%, at least greater than about 85%, at least greater than about 90%, or at least greater than about 95%.

The term “coating”, as used herein, refers to a layer or film that covers a surface of a substrate. This is the generic class for impregnating a base by causing a coating material to extend or penetrate into the base material, or into the interstices of a porous, cellular or foraminous material. Throughout this class, the term “coating” is used in the generic sense to include both surface coating and impregnation.

The coating may be hard or soft, permanent or transitory, supplied solely by extraneous materials or supplied wholly or in part by the base material.

The term “chemical vapor deposition (CVD)”, as used herein, refers to a chemical process which produces thin films. Typically, during CVD, precursors may react and/or decompose on the substrate surface to produce the film deposit. In certain embodiments, graphene can be formed in thin film using CVD.

The term “ocular”, as used herein, is pertinent or connected to eyes and vision. The term “ocular device” as used herein, refers to an object that can replace, assist or improve function of eye or eyesight, and the ocular device may be used in any part in the eye and nerve systems relating to eye functions or eyesight.

The term “prosthesis”, as used herein, refers to an artificial or synthetic device that can replace a body part. The prosthesis is particularly manufactured or adapted to replace or assist any missing or defective natural body parts for functional or cosmetic improvements. The prosthesis can be at least a portion of the body part or the entire body part, and can be used internally, partially embedded, or externally, without limitations to shape, size, material, and purpose of use.

The term “subject”, as used herein, refers to a mammal, including, but not limited to, human or human patient, to which a treatment or a surgery is performed for any medical or therapeutic reasons.

The term “surgery”, as used herein, refers to any operations aiming to treatment of injuries or disorders to any body parts by incision or manipulation, especially with use of an instrument.

The term “implant”, as used herein, refers to a performance, particularly a surgical performance to insert or replace a body part with an artificial object, prosthesis, graft, living tissue or hybrid thereof.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### BRIEF DESCRIPTION OF THE DRAWINGS

FIG. 1A depicts an exemplary disc substrate (PDMS/PMMA) coated with graphene using CVD; FIG. 1B depicts an exemplary disc (PDMA/PMMA) coated with graphene using 2 mL of graphene spray ink; FIG. 1C depicts an exemplary disc (PDMS) coated with graphene using 2.5 mL of graphene spray ink; FIG. 1D depicts an exemplary disc (PDMA/PMMA) coated with graphene using 3 mL of graphene spray ink; and FIG. 1E depicts an exemplary substrate (PDMA/PMMA) for producing an exemplary disc. For FIGS. 1B-1D, different amounts of graphene inks (graphene ink concentration 0.5 mg/mL) were used on the PDMS discs to check which concentration and volume ink was the one that, covering the entire surface of the disc, present the best transparency levels (this concentration-volume was applied to the rest of the substrate materials) (n=3).

FIG. 2A shows the diffusion transmittance of the exemplary discs (PMMA) coated with graphene using graphene ink and CVD; FIG. 2B shows the diffuse reflectance of the exemplary discs (PMMA) coated with graphene using graphene ink and CVD; FIG. 2C shows the absorption coefficient of the exemplary discs (PMMA) coated with graphene using graphene ink and CVD; and FIG. 2D shows the reduced scattering coefficient of the exemplary discs (PMMA) coated with graphene using graphene ink and CVD. The results in FIGS. 2A-2D were obtained by applying the Inverse Adding-Doubling (IAD) technique (n=3) and PMMA-CTR refers to a control without graphene coating.

FIG. 3 shows topographic evaluations of titanium discs (Ti) and titanium discs coated with 2 ml, 2.5 ml or 3 ml of graphene ink, or CVD graphene (n=3) by atomic force microscopy evaluation. No significant differences were found (p<0.05).

FIG. 4 shows cytotoxicity levels (spectrophotometry units) of human corneal fibroblast cultured on top of the petri dish (Petri), Titanium discs (Ti), or titanium discs coated with 2 ml, 2.5 ml or 3 ml of graphene ink, or CVD graphene (n=6). LDH is the positive control (all the samples were significantly different to the positive control, P<0.05%). Bars from left to right present Petri; Ti without graphene film; G1 coated using 2 ml of graphene ink; G2 coated using 2.5 ml of graphene ink; G3 coated using 3 ml of graphene ink; CVD coating; LDH positive sample, respectively.

FIG. 5 shows proliferation levels (ratio of the spectrophotometry units of the sample divided by the petri's value) of human corneal fibroblast cultured on top of Titanium discs (Ti), or titanium discs coated with 2 ml (G1), 2.5 ml (G2) or 3 ml (G3) of graphene ink, or CVD graphene, at 4 days (n=6). The ratio of the petri's proliferation is 1. No significant differences were found except between the groups G3 and CVD (p<0.05%).

FIG. 6 shows phase contrast pictures (4×) of human corneal fibroblasts cultured on top of CVD graphene (on top of PDMS substrate), non-coated PDMS or petri dish, at 24, 48, 72, 96 hours and 9 days.

FIG. 7 shows phase contrast pictures (4×) of human conical epithelial cells cultured on top of CVD graphene (on top of PDMS substrate), non-coated PDMS or petri dish, at 24, 48, 72, 96 hours and 9 days.

FIG. 8 shows LiveDead assay (10× pictures) of human conical fibroblasts cultured on top of CVD graphene or graphene ink (on top of PDMS substrate), non-coated PDMS or petri dish, at 24, 48, 72 and 96 hours.

FIG. 9 shows rose bengal (RB) uptake assays carried out to confirm the presence of barrier function in stratified epithelial cells cultured on top of CVD graphene, graphene ink, PDMS and petri dish. After staining with 0.1% RB, images were immediately photographed at room temperature with a 10× objective on a Nikon Inverted Eclipse TS100 microscope. Stratification, as shown by areas of RB exclusion, was detected in all the samples.

FIG. 10 shows histological evaluation based on methacrylate processing and H&E staining of stratified epithelial cells cultured on top of CVD graphene, graphene ink or non-coated PDMS discs.

FIG. 11 shows stratified human corneal fibroblast on top of a CVD graphene film (PDMS substrate) observed with Rose Bengal staining.

FIG. 12 shows stratified human corneal fibroblast on top of a graphene ink film (PDMS substrate) observed with methacrylate-based histology.

FIG. 13 shows results from a cell proliferation assay that was evaluated by MTS assay in 3 different cell culture populations of primary corneal fibroblasts obtained from 3 different human cornea donors, cultured on top of PDMS discs, at 24, 48, 72 and 96 hours after seeding. Results are displayed as mean +/−SD (N=9). Results are displayed as mean +/−SD (N=9).Significance was determined using one-way ANOVA with Bonferroni's post-hoc test. *p<0.05; **p<0.01; ***p<0.001. ns, non-significant.

FIG. 14 shows results from a cell proliferation was evaluated by MTS assay in 3 different cell culture populations of primary scleral fibroblasts obtained from 3 different human cornea donors, cultured on top of PDMS discs, at 24, 48, 72 and 96 hours after seeding. Results are displayed as mean +/−SD (N=9).Significance was determined using one-way ANOVA with Bonferroni's post-hoc test. *p<0.05; **p<0.01; ***p<0.001. ns, non-significant.

FIG. 15 shows transmission electron microscopy (TEM) evaluation of stratified human corneal-limbal epithelial cells cultured on top graphene coated PDMS according to an exemplary embodiment of the present invention.

FIG. 16 shows transmission electron microscopy (TEM) evaluation of stratified human corneal fibroblasts cultured on top CVD graphene coated PDMS according to an exemplary embodiment of the present invention.

FIG. 17 shows transmission electron microscopy (TEM) evaluation of stratified human corneal fibroblasts cultured on top ink graphene coated PDMS according to an exemplary embodiment of the present invention.

FIG. 18 shows a scratch assay on graphene coated PDMS disks.

### DETAILED DESCRIPTION OF THE INVENTION

The present invention provides a biointegrative and biocompatible medical device. In particular, the medical device of the invention comprises a graphene coating disposed on at least one surface of a substrate. The graphene coating features transparency, antibacterial properties, resistance, elasticity, biocompatibility, cell culture support, and conductivity, which may be most suitable for the biocompatible medical device.

### Device

In one aspect, the present invention provides a device or a medical device which comprises: a substrate and a graphene coating. In particular, the graphene coating is formed to be substantially transparent or transparent, by adjusting, for example, thickness, number of graphene layer, deposition density or area density thereof. In addition, the graphene coating is formed to have a thickness less than about 20 μm.

In some embodiments, at least one bonding layer may be disposed between the graphene coating and the substrate, without any limitations to a number of layers, a thickness thereof, or materials used in the bonding layers. Alternatively, at least one intermediate layer may be disposed between the graphene coating and the substrate, without any limitations to a number of layers, thickness thereof, or materials used in the those layers.

The graphene coating may be disposed or deposited on at least a portion of the surface, at least one surface of the substrate, or entire surface of the substrate, without any limitation. In certain embodiments, when the substrate is formed in a plane, a disc, a ring, a semi-ring, a cylinder, a sphere, a semi-sphere or any combinations thereof, the graphene coating may be disposed or deposited on entire or at least a portion of surface of the substrate.

The graphene coating can be applied by any coating methods known to one of ordinary skill in the arts. Exemplary coating method includes spraying, painting, immersing, electroplating, chemical vapor deposition (CVD), physical vapor deposition (PVD), chemical coating, anodizing, vacuum plating, dipping, thermal spraying, and the like.

Preferably, the graphene coating may be deposited by chemical vapor deposition (CVD). Generally used CVD may be suitably used by varying each parameter thereof, and the parameter may include a pressure, a type of reactor, a temperature, accelerating voltage, carrier gases, and the like. Exemplary CVD may also suitably adopt a low pressure CVD (LPCVD) or a ultra-high vacuum CVD (UHCVD), an atmospheric CVD including plasma-enhanced CVD (PECVD) or plasma assisted CVD (PACVD) and the like, but the examples are not limited thereto.

Preferably, the graphene coating formed by the CVD may suitably have a thickness less than about 10 μm, less than about 5 μm, less than about 1 μm, less than about 100 nm, or less than about 10 nm.

Alternatively, the graphene coating may be disposed on the substrate with an ink composition comprising graphene. The ink composition can be applied by spraying, painting, dipping, and the like. The ink composition may be suitably prepared based on the application or coating methods, and the ink composition may be further processed, treated or diluted with a solvent before application. For example, the ink composition used for spraying may comprise graphene in an amount of about 1 to about 50 wt % based on the total weight of the ink composition.

The graphene coating formed from the ink composition may suitably have a thickness less than about 100 μm, less than about 50 μm, less than about 40 μm, less than about 30 μm, less than about 20 μm, less than about 10 μm or less than about 5 μm, less than about 1 μm, less than about 500 nm less than about 250 nm, less than about 125 nm, less than about 100 nm, or less than about 10 nm. In an exemplary embodiment, the graphene coating can be formed by spraying the ink composition at a thickness less than about 20 μm, less than about 10 μm or less than about 5 μm, or less than about 1 μm.

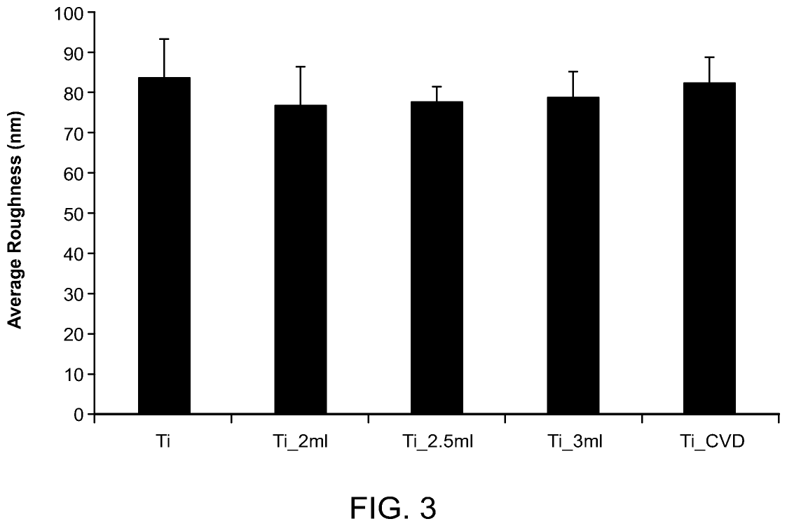

The graphene coating may be formed in a single layer or in multiple layers of graphene. Meanwhile, a surficial feature or topography of the substrate may not be altered or modified by the graphene coating. For example, as shown in FIG. 3, a titanium substrate, which is either coated with graphene by CVD or with the graphene ink composition, still maintains a normal nanotopography of the titanium (e.g. p<0.05).

In certain embodiments, the device may be an ocular device such as corneal device, intrastromal corneal ring segments, corneal inlays, glaucoma valves, intraocular biosensors or image processor, and the like. The ocular device may be transplanted by a surgical procedure or any treatments by incision or manipulation. In particular embodiments, the ocular device requires substantially high light transmittance thereof including the substrate and the coating, particularly within the visible light wavelengths.

In preferred embodiments, the graphene coating, as being coated on the substrate, may have a transmittance greater than about 10%, greater than about 20%, greater than about 30%, greater than about 40%, greater than about 50%, greater than about 60%, greater than about 70%, greater than about 80%, or particularly greater than about 90%.

In particular embodiments, when the graphene coating is formed by CVD coating, the graphene coating may have a transmittance greater than about 85%, greater than about 90%, or particularly greater than about 95%.

In preferred embodiments, the substrate may be transparent, or substantially transparent, having a transmittance greater than about 80%, greater than about 85%, greater than about 90%, or greater than about 95%. In particular embodiments, the substrate may be made of transparent or substantially transparent materials.

In preferred embodiment, the device may be transparent, or substantially transparent, having a transmittance greater than about 80%, transmittance greater than about 85%, greater than about 90%, or greater than about 95%.

The substrate may be formed of ceramics such as glass, polymer, composite, and mixtures thereof. In certain embodiments, the ceramic material that can be suitably used as the substrate for ocular devices may include (poly)crystalline transparent metallic ceramics, such as alumina Al₂O₃, yttria alumina garnet (YAG), neodymium-doped YAG, and transparent amorphous ceramics (e.g. glass).

The polymer that can be suitably used as the substrate for ocular devices may include synthetic polymers, for example, as silicone polymers, poly methyl methacrylate (PMMA), polydimethylsiloxane (PDMS), poly(c-caprolactone) (PCL), polyethylene glycol (PEG), polyethylene (glycol) diacrylate (PEGDA), polyglycerol sebacate (PGS), poly(1-lactide-co-d,1-lactide), poly (ester urethane) urea, poly(prolyl-hydroxyprolyl-glycyl), polymethacrylate hydrogel, polymethacrylic acid-co-hydroxyethyl methacrylate (PHEMA/MAA) hydrogel, poly(2-hydroxyethyl methacrylate-co-methacrylic acid), 2-hydroxyethylmethacrylate crosslinked hydrogel, N-ethyl-N-(3-dimethyl aminopropyl)carbodiimide/N-hydroxysuccinimide, polyvinylalcohol (PVA), polyvinylpyrrolidone, poly(3-hydroxybutyric acid-co-3-hydroxyvaleric acid) and the like, the examples are not limited thereto.

The polymer also may include biological polymers comprising carbohydrates, peptides, lipids and combinations thereof, for example, peptides such as silk and collagen (e.g. gelatin, collagen from humans or animals, from fish scales, electrospun collagen, and the like), cellulose, N-isopropylacrylamide, fibrin, keratin, chitosan (e.g. hydroxyethyl chitosan, hydroxypropyl chitosan, carboxymethyl-hexanoyl chitosan or the like), chondroitin (e.g. hydroxyapatitechondroitin, chondroitin sulfate or the like), polymethylmethacrylate, keratocytes, laminin, retinoic acid (RA), tobramycin, dextran, alginate, hyaluronan, lactone, polypyrrolidine, phospholipid (e.g. phosphatidylcholine such as lecithin, 2-methacryloyloxyethylphosphorylcholline and the like, phosphoinositides, sphingomyelin, and the like), bioactive peptide nanofibers, and derivatives thereof, but the examples are not limited thereto.

In some embodiments, the above described polymeric or biological polymeric materials can be cross-linked or combined without limitations to molecular weight or the like.

In some embodiments, the substrate may optionally include at least a portion surrounding (skirting) the optical part to improve biointegration of the device with the host tissue. Those skirting materials may be transparent or opaque. The portion may include metallic components including titanium, gold, silver, copper, or an alloy metal, ceramic, carbon components, or polymers. Exemplary materials used for the skirting portion metal components are listed in the following Table 1 (Reham et al, Materials, 2015, 8, 932-958).

TABLE 1

| | |
|---|---|
| Implant Material | Common Name or Abbreviation |

I. Metals

| | |
|---|---|
| Titanium | CpTi |
| Titanium Alloys | Ti—6Al—4V extra low interstitial (ELI) |
| | Ti—6Al—4V |
| | Ti—6Al—7Nb |
| | Ti—5Al—2.5Fe |
| | Ti—1 [symbol] Zr—4Nb—2Ta— [symbol] 2Pd |
| | Ti—29Nb—13Ta—4.6Zr |
| | [symbol] (83%-87%Ti—13%-17%Zr) |
| Stainless Steel | [symbol] 6 L [symbol] |
| Cobalt Chromium Alloy | Yitallium, Co—Cr—Mo |
| Gold Alloys | Au Alloys |
| Tantalum | Ta |

II. Ceramics

| | |
|---|---|
| Alumina | Al₂O₃, polycrystalline alumina or single- |
| | crystal sapphire |
| Hydroxy [symbol] | [symbol] , (OH)₂ |
| Beta-Tricalcium | β-TCP, C [symbol] (PO₄)₂ |
| Phosphate | |
| Carbon | C |
| | vitreous, |
| | low-temperature isotropic (LTI). |
| | ultra-low-temperature isotropic (ULTI) |
| Carbon-Silicon | C—Si |
| Bioglass | SiO₂/CaO/Na₂O/P₂O₅ |
| Zirconia | ZrO₂ |
| Zirconia-toughened alumina | ZTA |

III. Polymers

| | |
|---|---|
| Polymethylmethacrylate | PMMA |
| Polytetrafluoroethylene | PTFE |
| Polyethylene | PE |
| Polysulfone | PSF |
| Polyurethane | PU |
| Polyether ether ketone | PEEK |

Adopted from: Williams, 1981 [5]; Lemons, 1990 [6]; Craig, 1993 [7]; Sagomonyants et al., 2007 [8]; Berner et al., 2009 [9]. [symbol]
indicates data missing or illegible when filed

The substrate may not be limited in shape, size or thickness. In preferred embodiments, the substrate may be formed in an entire shape of the medical device, or prosthesis for implanting, or at least a portion thereof. For example, the substrate that can be used as the corneal device may have a shape of cornea, corneal ring, and the like, or a partial structure thereof. In preferred embodiments, the substrate may include a portion of prosthesis to be implanted, particularly the portion where host tissue can adhere and grow.

In an exemplary embodiment, the ocular device may be an artificial cornea or keratoprosthesis, having a shape of cornea, or a partial structure thereof.

In an exemplary embodiment, the ocular device may be a graphene intrastromal corneal ring, intrastromal layers or corneal inlays and the like for reshaping the cornea. For example, the graphene intracorneal ring may be manufactured by coating a ring shaped (skirt) PMMA, or titanium substrate with graphene, which is for correcting refractive errors and contact lens adaptation in a subject with corneal ectasia based on the modification of the corneal shape and curvature. The conventional non-coated PMMA ring, in contrast, has been reported to induce inflammation in the host cornea and major complication due to lack of biointegration.

In an exemplary embodiment, the present invention provides an intraocular lens (IOL) coated with the graphene. Preferably, the graphene coating may suitably have a thickness of the graphene coating less than about 1 μm, and may be totally transparent with the transmittance thereof greater than about 85%.

In an exemplary embodiment, other ocular prosthesis device such as a glaucoma drainage device, scleral buckles or retinal prosthesis can be manufactured by coating at least a portion or entire surface of the substrate with graphene to improve biointegration and biocompatibility thereof.

In preferred embodiments, the graphene coating, as being coated on the substrate, of the present invention can promote cell proliferation. The cell may include any cells from bacteria or prokaryote and eukaryotic cells. In certain embodiments, the mammalian or human cell can be cultured on the graphene coating.

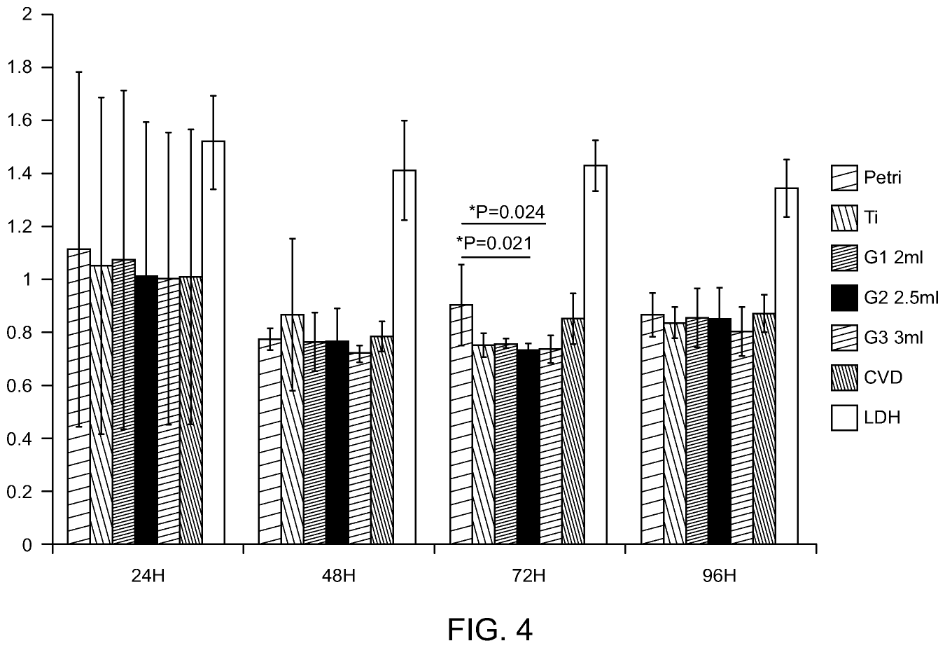

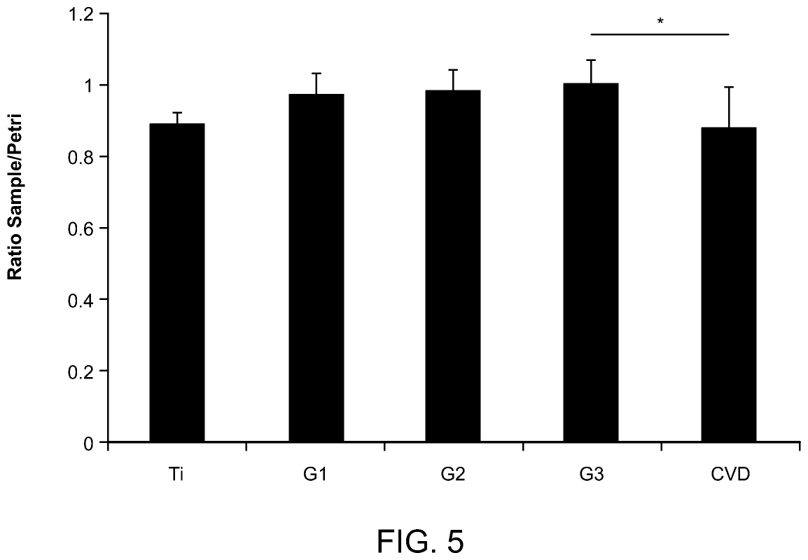

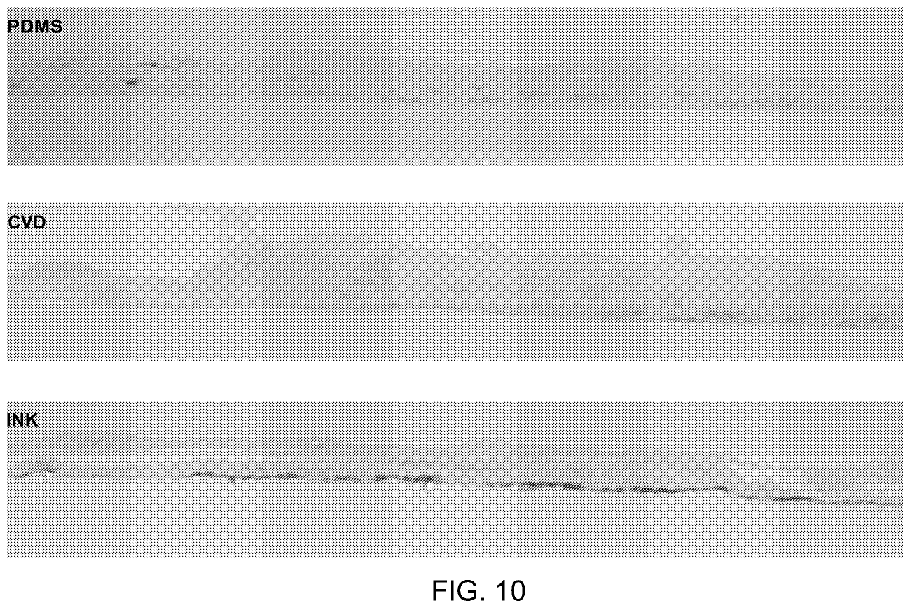

For example, different types of human corneal cells, (i) epithelial cells and (ii) fibroblasts were cultured on the graphene coating on various substrates and viability thereof was evaluated (FIGS. 4-5: graphene coating on a titanium substrate; and FIGS. 10-12: graphene coating on a PDMS substrate). The human corneal limbal epithelial cells and the human corneal fibroblasts can grow and spread when they are cultured on top of a well-defined graphene film formed by CVD or ink composition in a similar way to a petri dish, without limitations to the material used as a substrate where the graphene film is coated.

Meanwhile, non-coated materials like PDMS (silicone) did not optimally promote the cell adhesion of corneal fibroblasts in a similar way to a petri dish.

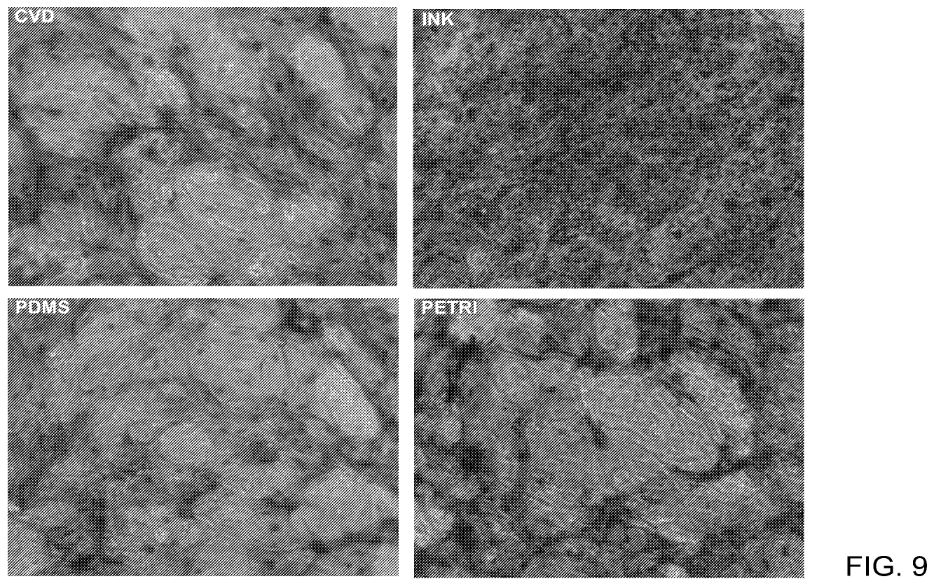

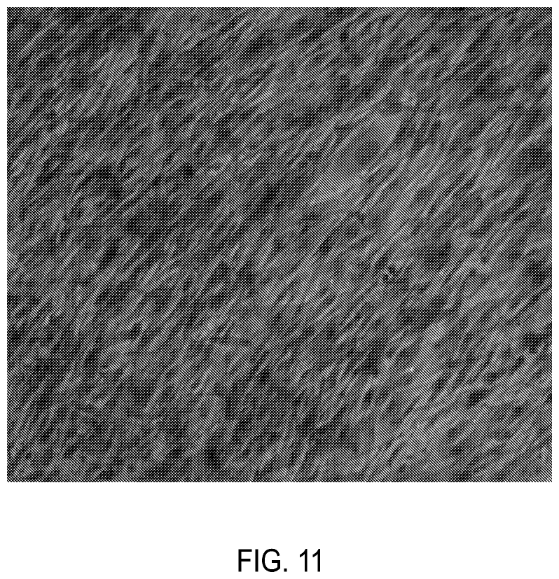

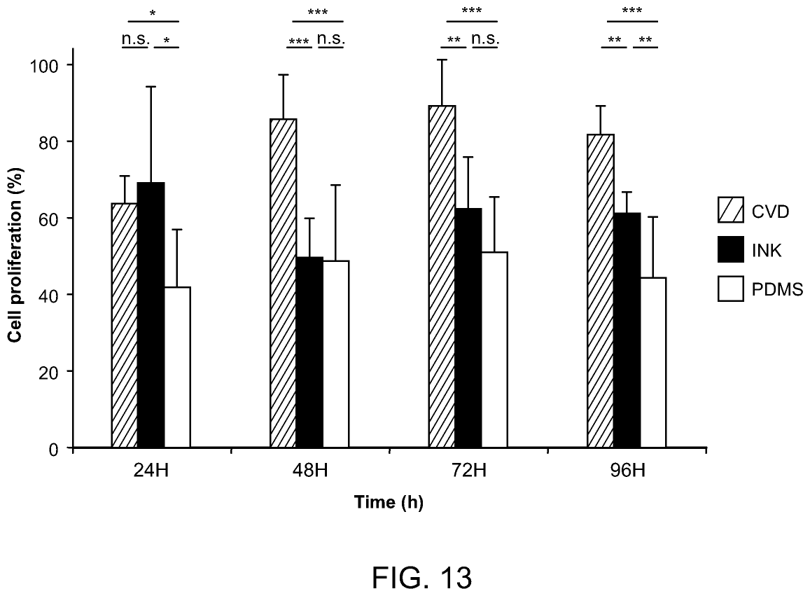

Moreover, the graphene coating, as being coated on the substrate, can promote cell differentiation as well as stratification of the cultured cells (FIGS. 9-12). For example, as shown in FIG. 9, differentiation assays based on the promotion of the stratification of the corneal cells were on PDMS constructs, which includes CVD graphene coated PDMS, ink graphene coated PDMS substrate, and non-treated (coated) PDMS substrate, and a petri dish. In each window in FIG. 9, rose bengal (RB) uptake assay demonstrated the existence of a barrier function following stratification on top of the graphene coated PDMS substrate, similar to the petri dish. The stratification was also demonstrated in FIG. 10. As shown in FIG. 10, the fibroblasts were incubated, and it was confirmed that stratification was promoted on graphene coating coated on the PDMS substrate. Further, RB and methacrylate-based histology were applied (FIGS. 11-17) and well-developed stratification of the cultured fibroblast was observed with the graphene coatings formed with CVD or ink spraying techniques on the PDMS substrates.

In certain embodiments, the medical device may further comprise an external device, sensor, circuit, central processing unit (CPU), screen-based device, antennae, near field communication circuit, wireless power source and the like.

In certain embodiments, the graphene coating, as being coated on the substrate, may include a sensor unit such as pH sensor or intraocular pressure (IOP) sensor, a signaling unit, a microchip, or a biomarker. For instance, microchips, computer processing units or other elements can be embedded and serve as a unit that displays or transport information directly the external devices, other medical devices embedded in the subject's body, nerve system or brain of the subject's body, and the like.

Further, the graphene coating, as being coated on the substrate, may be doped with ions, metals, small molecules, drugs, biomolecules such as DNA, RNA, or proteins, and the like. In addition, since the graphene has ion conductivity, electric conductivity and thermal conductivity, the graphene coating can be connected to the external device, sensor, circuit, central processing unit (CPU), screen-based device, antennae, near field communication circuit, wireless power source and the like.

### Methods

In another aspect, the present invention provides various applications and methods using the device or the medical device as described above.

In one embodiment, the present invention provides a method of manufacturing a prosthesis or a device. The method comprises coating a substrate for the prosthesis or the device with the graphene as described above. In particular embodiments, the graphene may be coated by CVD, and the CVD graphene coating may suitably have a thickness of less than about 10 μm, less than about 5 μm, less than about 1 μm, less than about 500 nm less than about 250 nm, less than about 125 nm, less than about 100 nm, or less than about 10 nm as described above. Alternatively, the graphene coating may be formed using the graphene ink composition as described above, to suitably have a thickness of less than about 20 μm, less than about 10 μm, less than about 5 μm, or less than about 1 μm as described above.

The prosthesis can be any devices or artificial body parts used for a human body and human patient. In particular, the graphene coated prosthesis of the present invention may promote biointegration thereof after implanting or surgery, for example, by promoting cell proliferation of host cells, connective tissue, epithelial tissue, neurons, and other relative cells. Exemplary prosthesis or device to which the graphene may be applied may include hip replacement, heart pacemakers, pins, bone plates, screws, rods, wires, rib cages, spinal fusion cages, finger and toe replacements, cranio-facial prosthetics, dental prosthesis, vessel clips, breast implants, chips, auricular (ear), nasal, ocular, neck prosthesis, somato-prostheses and the like, and any prosthesis in needs of the graphene coating to improve biointegrity to host cells may be included in the invention.

In certain embodiments, the prosthesis or the device is an ocular device. In particular embodiment, the ocular device may be used as a corneal substitute or for restoring the ocular surface such as keratoprosthesis and may comprise a corneal device, such as intrastromal corneal ring segment, and corneal inlays, or other ocular device that improve or restore the function or the anatomy of any part of the eye such as a glaucoma valve, scleral substitute, iris prosthesis, intraocular lens, retinal implant and the like.

The ocular device, particularly a corneal device, is transparent or substantially transparent, and the light transmittance thereof may be greater than about 80%, greater than about 85%, greater than about 90%, or greater than about 95%. In particular, the graphene coating or film formed on the substrate has a transmittance greater than about 80%, greater than about 85%, greater than about 90%, or greater than about 95%. Further, the graphene coating used in the ocular device may suitably have a thickness less than about 10 μm, less than about 5 μm, or less than about 1 μm, and such graphene coating may be formed by CVD as described above. The graphene coating may be formed with the graphene ink composition as described above, the thus prepared graphene may suitably have a thickness less than about 20 μm, less than about 10 μm, less than about 5 μm, less than about 1 μm, less than about 500 nm less than about 250 nm, less than about 125 nm, less than about 100 nm, or less than about 10 nm.

In one embodiment, the present invention provides a method of promoting integration of a prosthesis or a medical device into a host tissue or host organ after implanting.

In one embodiment, the present invention provides a method of promoting host tissue adhesion or cell growth. The graphene coating deposited on the medical device (e.g. ocular device) or prosthesis device can promote proliferation or adhesion of host cell. As such, when the graphene coated substrate is included at least a part of the prosthesis for surgical implanting, e.g. ketoprosthesis, host cell adhesion and proliferation on the surface of the implanted device or prosthesis can be promoted and further reduce the incidence of major complications such as extrusion, infection or foreign body reaction.

In one embodiment, the present invention provides a method of providing cell delivery system using the graphene coated device. The method may further include culturing or growing other human tissues or cells, in vivo, in vitro, or ex vivo, such as connective tissues, epithelial cell, neurons or mesenchymal cells, on the graphene coating thereby promoting host tissue or cell growth without any cytotoxic effect. In particular embodiments, the method may comprise providing a cell carrier or delivery system for limbal stem cells, corneal endothelial cells, retinal pigment epithelial (RPE) cells, and the like which can be delivered in the implanted eye part of the subject with a regenerative or therapeutic purpose.

In one embodiment, the present invention provides a method of delivering a drug or any other active molecule. The method may further include the doping or functionalization of graphene with ions, metals, small molecules, drugs, biomolecules such as DNA, RNA, or proteins, and the like. Thus, graphene can be used as a release system for delivering specific drug into body parts that may be suffering from any disorders or injuries and in needs treatments thereof. Moreover, the functionalization of the graphene with active molecules such as DNA, RNA, proteins and the like, may facilitate the control of different cellular pathways to promote or inhibit any cellular response in the tissues, such as proliferation, differentiation, cell attachment, inflammation and the like.

In one embodiment, the present invention provides a method of measuring pH, intraocular pressure (IOP), different ions or molecules such as glucose, inflammatory markers, and the like by using the graphene coated device as describe above. In preferred embodiments, the device may further comprise an external device, a sensor, a circuit, and a central processing unit (CPU), screen-based device, antennae, near field communication circuit, wireless power source and the like. Further, in preferred embodiments, the graphene coating, as being coated on the substrate, may include a sensor unit such as pH sensor or intraocular pressure (IOP) sensor, a signaling unit, a microchip, a transmitter, or a biomarker. In addition, the graphene coating, as being coated on the substrate, may be doped with ions, metals, small molecules, drugs, biomolecules such as DNA, RNA, or proteins, and the like. In certain embodiments, the graphene coating, as being coated on the substrate, can be connected to the external device, sensor, circuit, central processing unit (CPU), screen-based device, antennae, near field communication circuit, wireless power source and the like.

### EXAMPLE

### Example 1. Optical Evaluation of Graphene Coated Disc

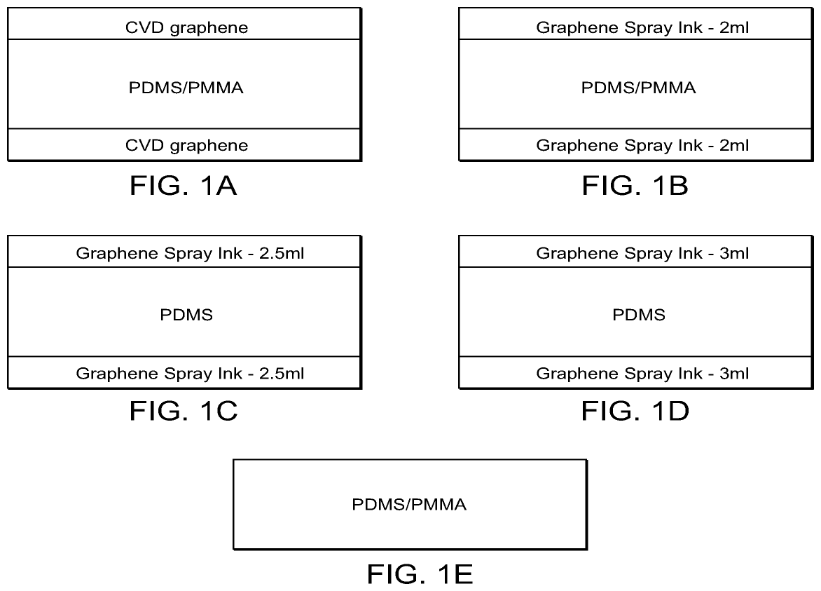

Both surfaces of a disc were coated with graphene by CVD or graphene ink (FIG. 1) and four sample species as shown in FIG. 1 were prepared. In panel A, the graphene coating was formed on both sides of the PDMS/PMMA substrate by CVD deposition; in panel B, the graphene coating was formed on both sides of the PDMS/PMMA substrate using 2 mL of the graphene spray ink; in panel C, the graphene coating was formed on both sides of the PDMS substrate 2.5 mL of the graphene spray ink; and in panel D, the graphene coating was formed on both sides of the PDMS substrate 3 mL of the graphene spray ink.

For the optical evaluation, the Inverse Adding-Doubling (IAD) technique was applied for evaluating the optical properties of an exemplary disc (PMMA) coated with graphene.

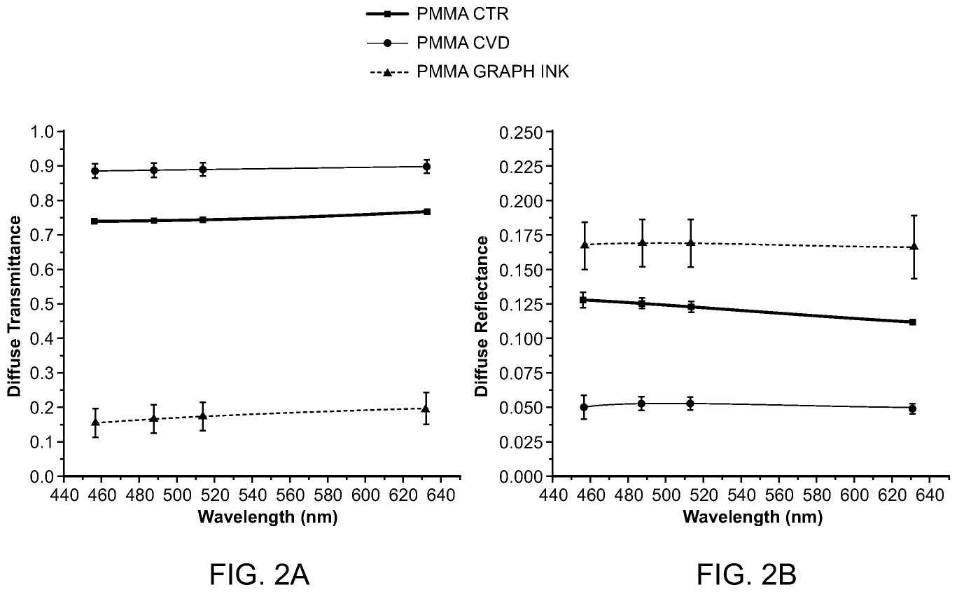

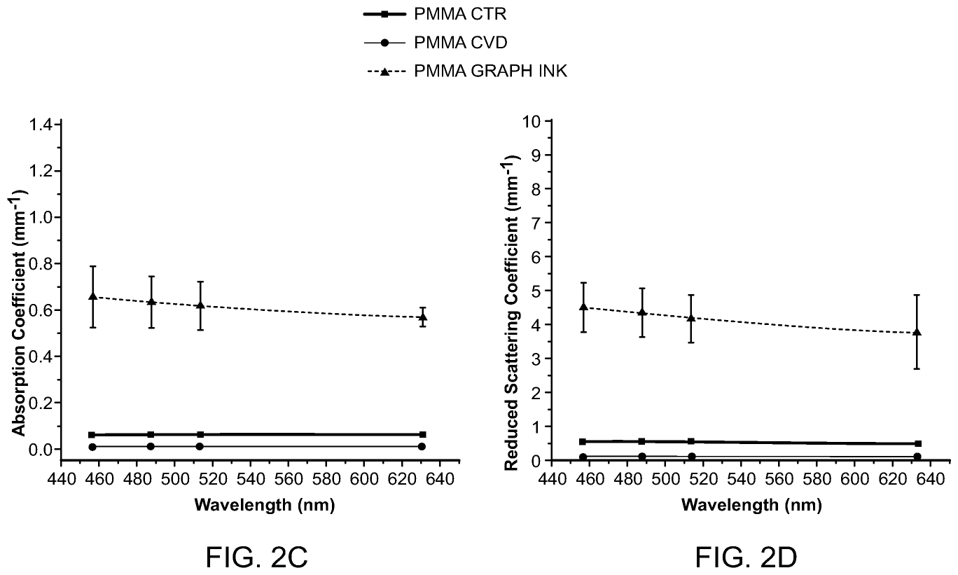

The results measuring diffuse transmittance, diffuse reflectance, absorption coefficient and reduced scattering coefficients are shown respectively in FIGS. 2A-2D. As shown in FIG. 2A, the CVD constructs always present a transparency level (diffuse transmittance) of greater than about 85%, similar to the controls (the substrate material without the graphene film such as FIG. 1E). However, the ink constructs presented a transparency level less than about 20%. For further details of the optical properties (transmittance, scattering, absorbance and reflectance levels) of each constructs depending on the type of substrate and graphene used (FIG. 2A-2D)

Since it has been demonstrated that the topography of the materials influences the growth and phenotype of the cells, the topographies of the coating materials used in FIG. 1 (CVD and ink graphene) were inspected to check whether topographies the surface of the substrate were changed by the deposition of those graphene coating. For that purpose, Atomic Force Microscopy Assay was carried out to evaluate each titanium discs coated with CVD or graphene ink (FIG. 3). As shown in FIG. 3, both types of graphene film coating did not alter the normal nanotopography of the titanium surface (p<0.05). Therefore, it can be confirmed that all the effects from the graphene coated devices or discs are associated to the graphene coating itself, not due to a change in the topography of the substrate.

### Example 2. Proliferation Capacity of Graphene Coating

The graphene coated substrates were evaluated for proliferative capacity and viability of human corneal cells cultured on top of graphene coatings performing different assays: MTS assay, LDH assay, Live/Dead assay and a microscopic cell covering surface analysis. Firstly, 15-mm diameter disks made of different substrates (e.g. PDMS, PMMA, or titanium) were coated with CVD or ink graphene. Afterwards, triplicates of each disk variation were placed in 24-well plates and seeded with different confluent human corneal cell cultures including (i) epithelial cells and (ii) fibroblasts (1×10⁴ cells/cm²). Cells were incubated in 37° C. humidified, 5% CO₂ atmosphere. The non-coated substrate disk served as material control, whereas positive and negative cell controls were used either by plating cells alone on the petri dish or using the feeding medium alone, respectively.

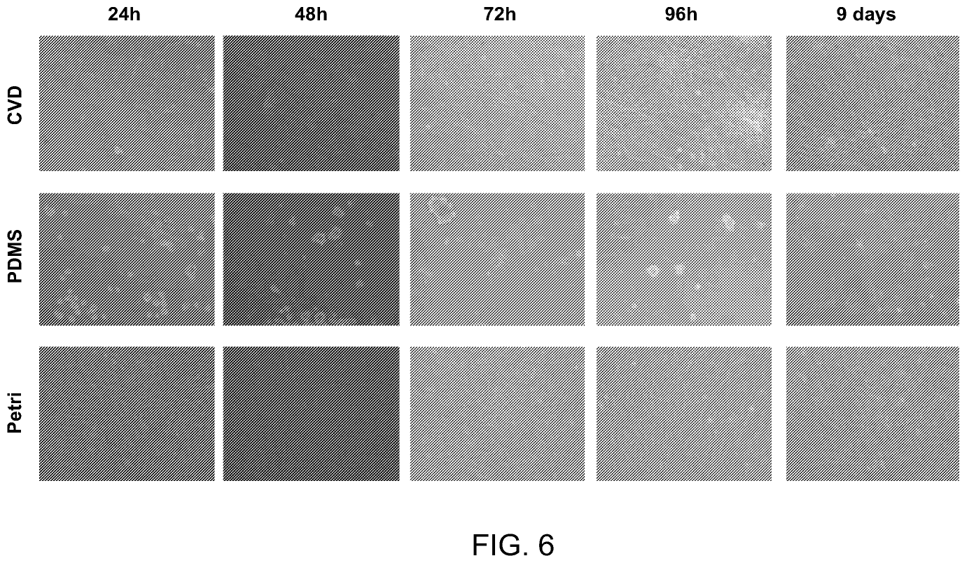

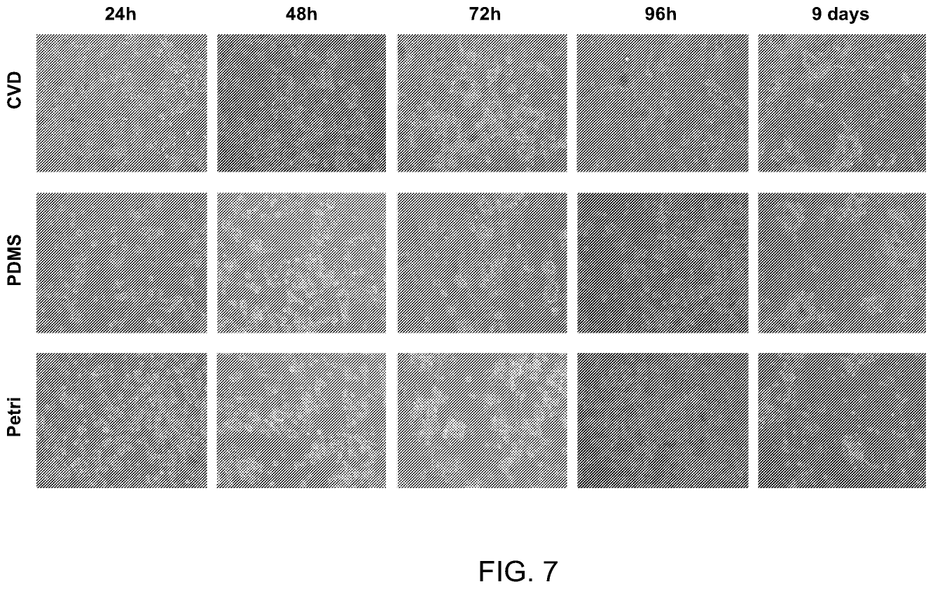

A microscopic cell covering surface analysis was carried out to evaluate the cell proliferation and migration level of cell populations on top PDMS (FIG. 6) as substrate which were coated with CVD graphene, the non-coated substrate and the petri dish. For that purpose, phase contrast images of cell cultures were taken at 24, 48, 72 and 96 hours after seeding with a 4× and 10× objectives using an inverted microscope (Nikon Eclipse TS100, Nikon Instruments Inc.; Melville, N.Y.).

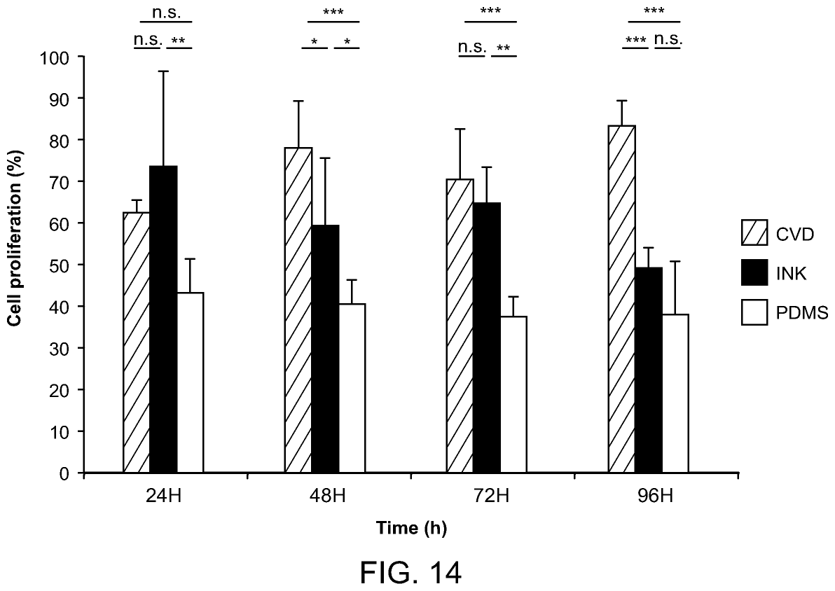

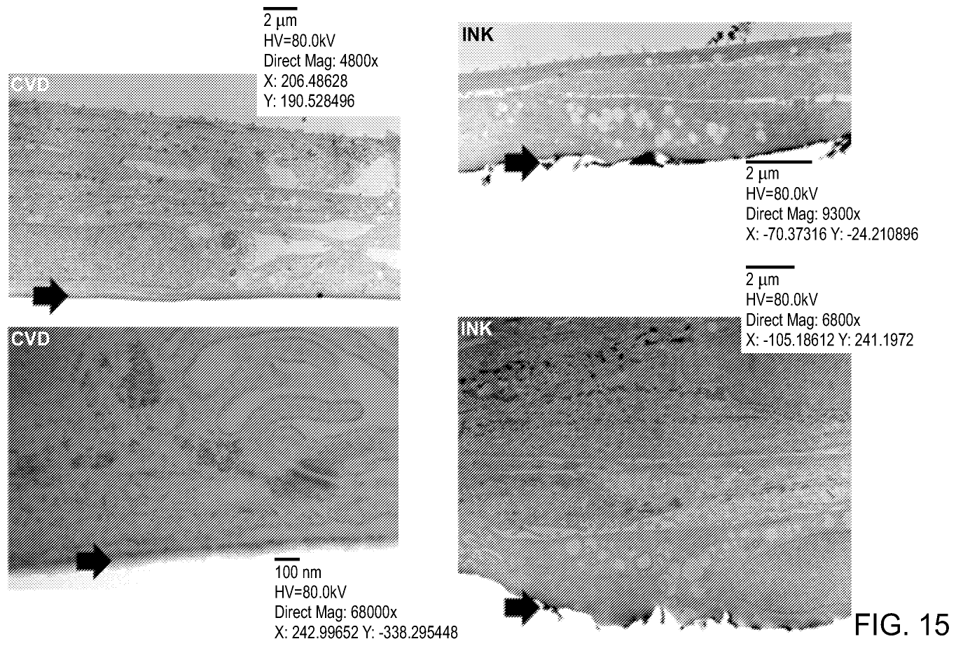

Cell proliferation (CellTiter 96® AQueous One Solution Cell Proliferation Assay; Promega, Madison, Wisc.) was evaluated by MTS assay in 3 different cell culture populations of primary corneal or scleral fibroblasts obtained from 3 different human cornea donors, cultured on top of PDMS discs, at 24, 48, 72 and 96 hours after seeding (n=9) (FIGS. 14-15). 15-mm diameter disks made of PDMS were coated with CVD or ink graphene. Afterwards, triplicates of each disk variation were placed in 24-well plates and seeded with 1×10⁴ fibroblasts per cm². Cell proliferation was normalized on human corneal fibroblasts cultured on a petri dish for the specific time point, arbitrarily set as 100%. The non-coated substrate disk served as material control, whereas positive and negative cell controls were used either by plating cells alone on the petri dish or using the feeding medium alone, respectively. Cell proliferation was normalized on human corneal fibroblasts cultured on a petri dish for the specific time point, arbitrarily set as 100%.

Furthermore, the same assay was performed using 3 different cell culture populations of primary scleral fibroblasts obtained from 3 different human cornea donors to evaluate any differences between different types of fibroblast that present different fibrotic phenotype based on the expression of alpha smooth muscle acting (FIG. 5).

Cytotoxicity (CytoTox 96® Nonradioactive Cytotoxicity Assay; Promega) was evaluated by LDH assay in 3 different cell culture populations of primary corneal fibroblasts obtained from 3 different human cornea donors, cultured on top of coated and non-coated PDMS disks, at 24, 48, 72 and 96 hours after seeding (n=9). LDH assay was also performed for coated and non-coated substrates composed by PMMA, Titanium, seeded with 2 different cell culture populations of primary corneal fibroblasts obtained from 2 different human cornea donors (n=6). Cytotoxicity levels were normalized on human corneal fibroblasts cultured on a petri dish for the specific time point, arbitrarily set as 100%. Only significant differences were found between the LDH positive control and the rest of the groups, finding no significant differences between the different substrates or the coatings. Moreover, LDH absorbance units were compared without normalization among titanium disks coated with 2 ml, 2.5 ml or 3 ml of graphene ink or CVD graphene (n=6) to evaluate possible differences in cytotoxicity derives from other ink concentrations. No significant differences were found among the different inks used (FIG. 5).

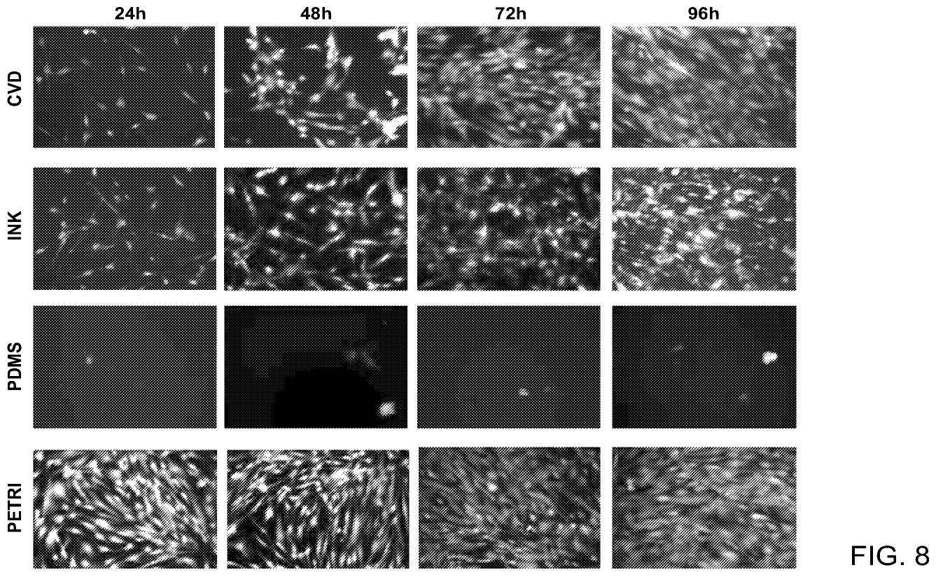

Furthermore, a Live/Dead assays were performed to evaluate the cell viability and the cell covering surface. Cells plated on top of the disks were incubated (without fixation) at room temperature for 30 minutes with a mixture of solution containing 2 μM calcium and 4 μM ethidium bromide prepared in PBS. Samples were observed under a Zeiss Axio Observer Z1 inverted fluorescent microscope (Carl Zeiss Microimaging GmbH, Jena, Germany) The assay results revealed that viable fibroblast perfectly grow on top of graphene (CVD or ink) on a similar way to the petri dish. However, fibroblasts were not able to grow properly on top of non-coated PDMS. Epithelial cells can grow on top of CVD or ink graphene; however, CVD graphene showed a better growth than the ink, similar to the controls (the non-coated substrate and the petri dish) (FIG. 8).

### Example 3. Differentiation Assay

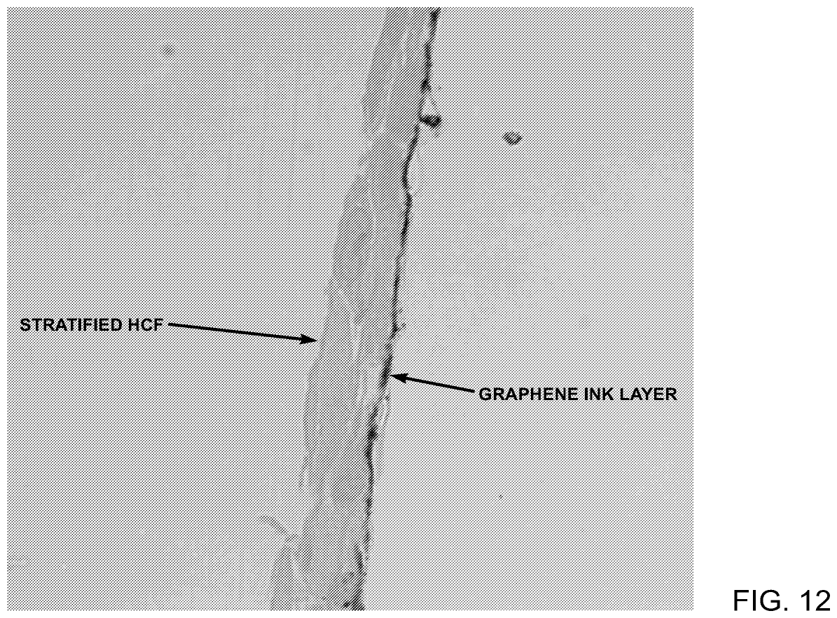

Differentiation assays were conducted based on the promotion of the stratification of the corneal cells on PDMS constructs. The epithelial cells were incubated for 7 days in Dulbecco's modified Eagle's medium (DMEM)/F-12 (Sigma-Aldrich) supplemented with 10% calf serum and 10 ng/ml epidermal growth factor to promote differentiation and stratification. A rose bengal (RB) uptake assay was carried out to confirm the presence of barrier function in stratified cells (FIG. 9). RB uptake assay demonstrated the existence of a barrier function following stratification on top of the graphene and non-coated PDMS, similar to the petri dish. The stratification was also demonstrated performing a histological evaluation based on methacrylate processing and haematoxylin and eosin (H&E) staining (FIG. 10). The fibroblasts were incubated for 30 days in Eagle's medium (EMEM) supplemented with 10% fetal bovine serum and 10 ng/ml ascorbic acid to promote stratification. RB and methacrylate-based histology were applied (FIGS. 11-12). A well-developed stratification of the cultured fibroblasts was observed with both techniques on top of the graphene films.

Further, FIG. 15 shows transmission electron microscopy (TEM) evaluation of stratified human corneal-limbal epithelial cells cultured on top graphene coated PDMS. Telomerase-immortalized human corneal-limbal epithelial cells were seeded (1×10⁴ cells/cm²) and grown in a stratified cell culture system (at 37° C. and 5% CO₂) on top graphene coated disks made of PDMS. 15-mm diameter disks made of PDMS were coated with CVD or ink graphene (black arrow, in the images shown). Cells were grown as monolayers in keratinocyte serum-free medium (K-SFM) (Life Technologies; Carlsbad, Calif.) to achieve confluence. Cells were then incubated in Dulbecco's modified Eagle's medium (DMEM)/F-12 (Sigma-Aldrich; St. Louis, Mo.) supplemented with 10% newborn calf serum (Thermo Scientific; Rockford, Ill.) and 10 ng/ml EGF (Life Technologies) for 7 days to promote stratification and differentiation. Afterwards, transmission electron microscopy (TEM) evaluation was performed. For that purpose, the samples were processed for TEM using standard procedures, and viewed and photographed with an electron microscope (Tecnai G2 Spirit: FEI Company; Hillsboro, Oreg.). Proper stratification of the corneal epithelium was observed together with the presence of differentiated features such as microvilli, in the surface of the outer cell layer, and intercellular junctions, for both type of graphene coatings. Furthermore, the epithelium grown on top of graphene ink showed an increase of intracellular vesicles.

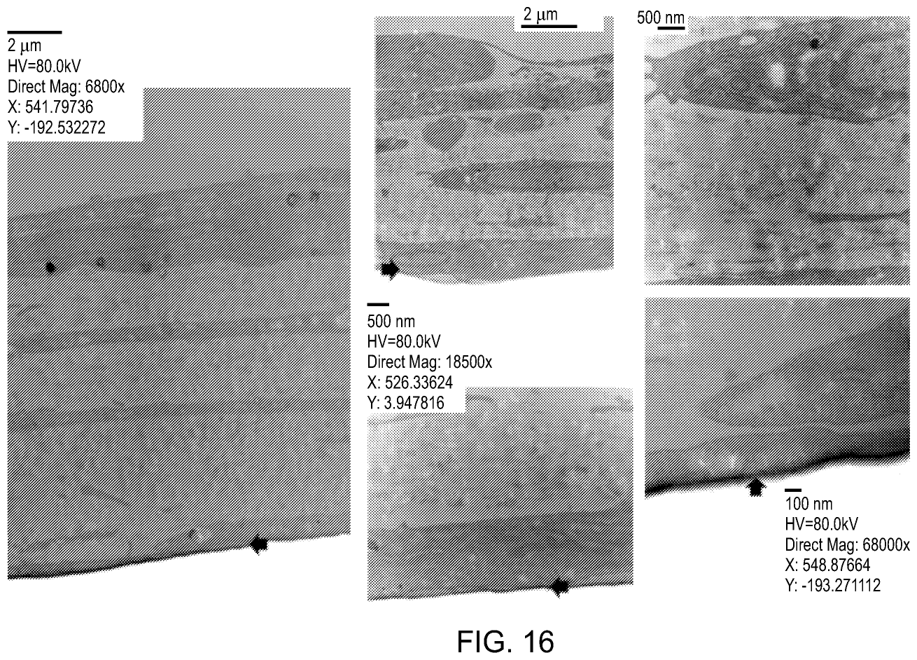

FIG. 16 also shows other transmission electron microscopy (TEM) evaluation of stratified human corneal fibroblasts cultured on top CVD graphene coated PDMS. Human corneal fibroblasts were seeded (1×10⁴ cells/cm²) and grown in a stratified cell culture system (at 37° C. and 5% CO₂) on top graphene coated disks made of PDMS. 15-mm diameter disks made of PDMS were coated with CVD graphene (black arrow, in the images shown). Cells were grown as monolayers in with Eagle's Minimum Essential Medium (EMEM: ATCC; Manassas, Va.) containing 10% fetal bovine serum (FBS: ATCC) to achieve confluence. Cells were then incubated in EMEM with 10% FBS and stimulated with Ascorbic acid for 30 days to promote stratification and differentiation. Afterwards, transmission electron microscopy (TEM) evaluation was performed. For that purpose, the samples were processed for TEM using standard procedures, and viewed and photographed with an electron microscope (Tecnai G2 Spirit: FEI Company; Hillsboro, Oreg.). A proper stratification of the corneal fibroblasts was observed together with the presence of orientated collagen lamellae between the cells.

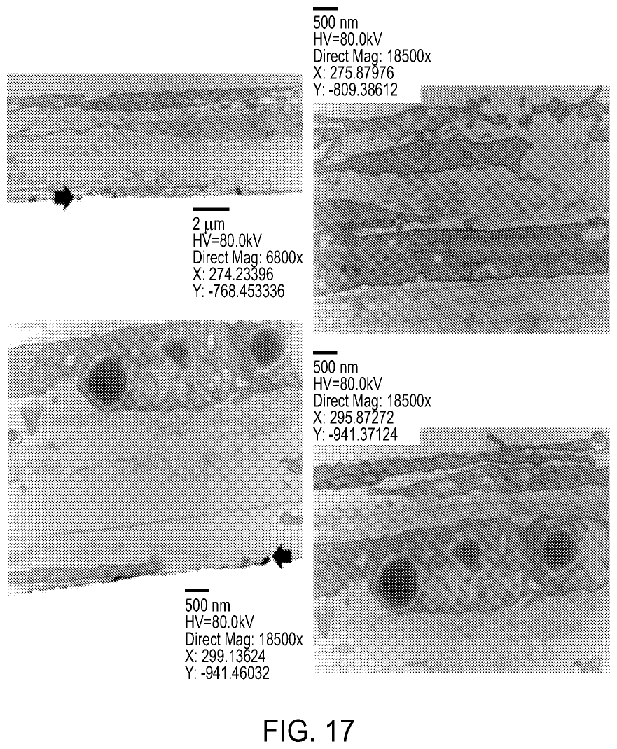

Additionally, FIG. 17 shows transmission electron microscopy (TEM) evaluation of stratified human corneal fibroblasts cultured on top ink graphene coated PDMS. Human corneal fibroblasts were seeded (1×10⁴ cells/cm²) and grown in a stratified cell culture system (at 37° C. and 5% CO₂) on top graphene coated disks made of PDMS. 15-mm diameter disks made of PDMS were coated with ink graphene (black arrow, in the images shown). Cells were grown as monolayers in with Eagle's Minimum Essential Medium (EMEM: ATCC; Manassas, Va.) containing 10% fetal bovine serum (FBS: ATCC) to achieve confluence. Cells were then incubated in EMEM with 10% FBS and stimulated with Ascorbic acid for 30 days to promote stratification and differentiation. Afterwards, transmission electron microscopy (TEM) evaluation was performed. For that purpose, the samples were processed for TEM using standard procedures, and viewed and photographed with an electron microscope (Tecnai G2 Spirit: FEI Company; Hillsboro, Oreg.). Proper stratification of the corneal fibroblasts was observed together with the presence of orientated collagen lamellae between the cells, on both type of graphene coatings.

Hence, the data demonstrate that graphene is an excellent candidate to be used to coat substrates used for a medical device, e.g. corneal devices or keratoprosthesis (PMMA, PDMS, and Titanium). Graphene is cell-friendly, promoting human corneal cells proliferation, viability and differentiation. The data also demonstrate that that CVD graphene is optically optimum, remaining transparent and not affecting the optical properties of the substrate used.

### Example 4. Scratch Assay

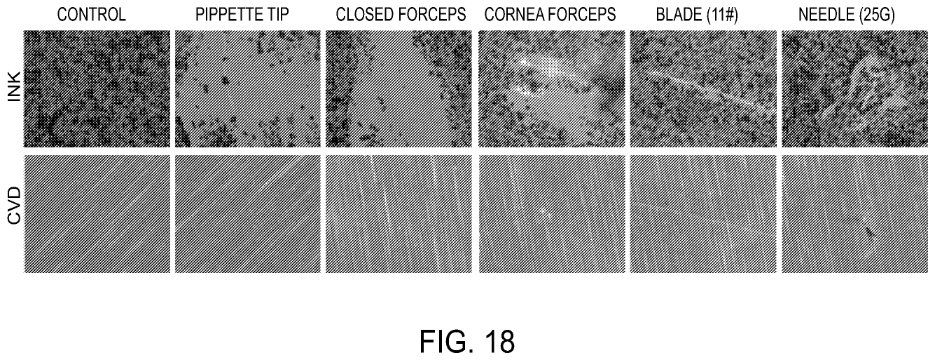

FIG. 18 shows results from a scratch assay on graphene coated PDMS disks. 15-mm diameter disks made of PDMS were coated with CVD or ink graphene. Afterwards, the samples were scratched with different methods: a lineal scratch with a 10-μl pipette tip, a lineal scratch with a closed forceps, grabbing the sample with a corneal forceps, a lineal scratch with a surgical blade (num. 11), or inserting a 25G-needle into the sample. Then, the integrity of the graphene coating was observed and imaged using phase contrast microscopy with a 4× objective in an inverted microscope (Nikon Eclipse TS100, Nikon Instruments Inc.; Melville, N.Y.). The results show that the CVD graphene remained almost intact for most of the scratch assays compared to the ink coating, which is easily detached from the PDMS.

### Example 5. Evaluation of Graphene as a Novel Material for Promoting Biointegration of Keratoprosthesis

The complication occurring in the implantation of keratoprosthesis (KPro) has been known to be associated at least to the lack of integration of the KPro materials surrounding corneal tissue. Accordingly, the conical biocompatibility of the graphene or graphene coating having an atomic layer-thick film of carbon atoms was tested and determined as a suitable coating material to improve the biointegration of KPro.

Different substrates (e.g., PDMS, PMMA, or titanium) were coated with graphene using chemical vapor deposition (CVD) or sprayed graphene ink. Optical evaluations of those samples were carried out based on the Inverse Adding-Doubling (IAD) technique.

In order to test the biocompatibility, thus prepared graphene coated substrates were also evaluated for proliferative capacity and viability of human corneal cells cultured on top of the graphene coatings. As such, different assays such as MTS assay, LDH assay, Live/Dead assay, microscopic cell covering surface analysis and evaluation of total protein concentration were performed.

In addition, differentiation assays were conducted based on the promotion of the stratification of the conical cells on graphene-coated PDMS as applying histological and ultrastructural evaluations as described above.

As results, the optical evaluation revealed that the CVD graphene coating did not affect the transparency of the different substrates used. However, sprayed graphene ink coating caused a significant decrease in transparency. Regarding proliferation and viability evaluation, human corneal cells spread and grew on top of the CVD graphene coating on a similar level to the positive control (a cell culture plate). Nevertheless, the graphene coating obtained from the sprayed graphene ink partially inhibited proliferation and cell viability. Regarding differentiation assays, histology and electronic microscopy evaluations showed that human corneal epithelial cells and fibroblasts were able to stratify on top of CVD graphene coating and graphene ink coating. Rose bengal uptake assay demonstrated the existence of a barrier function on the stratified epithelium cultured on top graphene.

Accordingly, the graphene coating formed by CVD process has been proved an excellent material (surficial material) due to its optical properties and high corneal biocompatibility shown in vitro. Therefore, the graphene coatings of the present invention, particularly the graphene coating formed by CVD, can be used as an ideal coating to improve the biointegration of KPro.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

### REFERENCES

Aldave et al., 2012. International results with the Boston type I keratoprosthesis. Ophthalmology, 119, 1530-8.

Aquavella et al., 2005. Keratoprosthesis: the Dohlman-Doane device. American journal of ophthalmology, 140, 1032-1038.

Dudenhoefer et al., 2003. Histopathology of explanted collar button keratoprostheses: a clinicopathologic correlation. Cornea, 22, 424-8.

Harissi-Dagher et al., 2007. Importance of nutrition to corneal grafts when used as a carrier of the Boston Keratoprosthesis. Cornea, 26, 564-8.

Linnola et al., 2003. Adhesion of soluble fibronectin, vitronectin, and collagen type IV to intraocular lens materials. Journal of cataract and refractive surgery, 29, 146-52.

Linnola et al., 2000a. Adhesion of fibronectin, vitronectin, laminin, and collagen type IV to intraocular lens materials in pseudophakic human autopsy eyes. Part 1: histological sections. Journal of cataract and refractive surgery, 26, 1792-806.

Linnola et al., 2000b. Adhesion of fibronectin, vitronectin, laminin, and collagen type IV to intraocular lens materials in pseudophakic human autopsy eyes. Part 2: explanted intraocular lenses. Journal of cataract and refractive surgery, 26, 1807-18.

Medeiros et al., 2014. Polymethylmethacrylate dermal fillers: evaluation of the systemic toxicity in rats. International journal of oral and maxillofacial surgery, 43, 62-7.

Moure et al., 2012. Clinical and pathological characteristics of polymethylmethacrylate and hyaluronic acid in the rat tongue. International journal of oral and maxillofacial surgery, 41, 1296-303.

Oliva et al., 2012. Turning the tide of corneal blindness. Indian J Ophthalmol, 60, 423-7.

Pascolini et al., 2010. Global estimates of visual impairment: 2010. Br J Ophthalmol, 96, 614-8.

Ruckhofer et al., 2000. Confocal microscopy after implantation of intrastromal corneal ring segments. Ophthalmology, 107, 2144-51.

Vargas et al., 2014. Local and systemic tissue response submitted to injection of 2 and 30% polymethylmethacrylate in rats' tongue. Gerodontology.

1. A medical device comprising,
a substrate; and
a graphene coating,
wherein the graphene coating is substantially transparent, wherein the graphene coating has a thickness less than about 20 μm,
wherein the graphene coating is disposed on at least one surface of the substrate.

2-8. (canceled)

9. The medical device of claim 1, wherein the graphene coating is formed in a film.

10. The medical device of claim 1, wherein the graphene coating comprises a single layer of graphene or multiple layers of graphene.

11. (canceled)

12. The medical device of claim 1, wherein the medical device is an ocular device.

13. The medical device of claim 12, wherein the graphene coating has a light transmittance greater than about 80% and the substrate has a transmittance greater than about 80%.

14-15. (canceled)

16. The medical device of claim 12, wherein the substrate comprises of ceramics polymer, composite, and mixtures thereof.

17. The medical device of claim 12, wherein the substrate is formed in a plane, disc, a ring, a semi-ring, a cylinder, a sphere, a semi-sphere or any combinations thereof.

18. (canceled)

19. The medical device of claim 1, wherein the medical device further comprises at least one of an external device, a sensor, a circuit, a central processing unit (CPU), screen-based device, antennae, near field communication circuit, and wireless power source and the graphene coating comprises a sensor unit, a microchip or a transmitter that is connected to an external device.

20. (canceled)

21. The medical device of claim 1, wherein the graphene coating comprises an ion, a biomolecule, a synthetic compound or a biomarker.

22. (canceled)

23. A method of manufacturing a medical device of claim 1, the method comprising:
depositing a graphene coating on a substrate,
wherein the graphene coating is substantially transparent, wherein the graphene coating has a thickness less than about 20 μm.

24. The method of claim 23, wherein the graphene is deposited by chemical vapor deposition (CVD) or spraying an ink composition comprising graphene.

25. (canceled)

26. A method of manufacturing a prosthesis comprising: coating a substrate of the prosthesis with graphene, wherein the coated graphene is substantially transparent and has a thickness less than about 20 μm.

27-31. (canceled)

32. A method of manufacturing an ocular device, comprising: coating a substrate of the ocular device with graphene, wherein the coated graphene is substantially transparent, wherein the graphene coating has a thickness less than about 20 μm.

33. The method of claim 32, wherein the ocular device is a keratoprosthesis, intrastromal corneal ring segment, and corneal inlays, a glaucoma valve, iris prosthesis, intraocular lens, scleral substitute, or retinal implant.

34. The method of claim 32, wherein the graphene is coated by CVD.

35-40. (canceled)

41. The method of claim 32, wherein the graphene has a transmittance greater than about 80%.

42-43. (canceled)

44. A method of promoting proliferation or cell adhesion of a cell on a prosthetic device, the method comprising: providing the device of claim 1 and contacting the device to an ocular cell.

45. The method of claim 44, wherein the graphene is coated by CVD or using an ink composition comprising graphene.

46-51. (canceled)

52. The method of claim 44, the cell is a host cell or host tissue, or an allogenic or xenogeneic cell.

53. (canceled)

54. The method of claim 44, wherein said cell comprises a human corneal limbal cell, a human limbal epithelial stem cell, a human corneal epithelial cell, or a human retinal pigment epithelial cell.

55. (canceled)

56. The method of claim 44, wherein said contacting step occurs prior to or after implanting said device into a subject.

57. (canceled)

58. The method of claim 44, wherein the device is an ocular device.

59. The method of claim 58, wherein the graphene coated on the ocular device has a transmittance greater than about 80%.

60-61. (canceled)

62. The method of claim 58, wherein the ocular device is a keratoprosthesis, an intrastromal corneal ring segment, a corneal inlay, a glaucoma valve, iris prosthesis, intraocular lens, scleral substitute, or retinal implant.

63. A method of for cell delivery system, the method comprising: providing the device of claim 1, said device further comprising an ocular cell.

64-68. (canceled)

69. A method of promoting biointegrating of a prosthesis or a device to a subject, the method comprising: coating the prosthesis or device with graphene, wherein the coated graphene is substantially transparent and has a thickness less than about 20 μm.

70. The method of claim 69, wherein the graphene is coated by CVD or using an ink composition comprising graphene.

71-75. (canceled)
